(19) United States
(12) Patent Application Publication
(10) Pub. No.: US 2004/0241827 A1
(43) Pub. Date: Dec. 2, 2004

(54) Adaptation of bacteria for use in leaching

(76) Inventors: Colin John Hunter, Dalkeith (AU); Tamsin Lisa Williams, Maylands (AU)

Correspondence Address: ROTHWELL, FIGG, ERNST & MANBECK, P.C., 1425 K STREET, N.W., SUITE 800, WASHINGTON, DC, 20005, US

(21) Appl. No.: 10/484,421

(22) PCT Filed: Jul. 19, 2002
(86) PCT No.: PCT/AU02/00971

(30) Foreign Application Priority Data
Sep. 3, 1999 (AU) PQ2651

(51) Int. Cl. C12P 3/00; C12N 1/21

(52) U.S. Cl. 435/252.3; 435/168

(57) ABSTRACT

A method for the adaptation of bacteria for use in the leaching of ores and concentrates, including the steps of: a) obtaining samples of bacteria exhibiting one or more desired attributes; and b) combining bacterial samples from step a) with a stock bacterial culture known to have the ability to oxidise sulphide minerals, whereby the resulting combined bacterial culture ultimately expresses both the one or more desired attributes and the ability to oxidise sulphide minerals. The particular desired attribute is preferably salt tolerance.

### FIELD OF THE INVENTION

The present invention relates to a method for the adaptation of bacteria for use in the leaching of ores and concentrates. More particularly, the method of the present invention relates to the adaptation of sulphide mineral oxidising bacterial cultures to operate effectively in specific environments, including saline environments.

### BACKGROUND ART

The bacterial oxidation of sulphide minerals requires reasonable volumes of process water whether leaching takes place in tanks, vats, dumps or heaps. The bacterial cultures used in these leaches operate well in waters with low total dissolved solids (TDS) and more importantly, low levels of chloride ions. In some areas of the world, particularly in Australia, good quality process water is very difficult to find and the cost of improving water quality through the use of water treatment plants is very high. In order to use bacterial leaching in these low quality process waters it is essential that sulphide oxidising bacteria are adapted to saline environments.

Bacteria are ubiquitous in the environment and can be found in such diverse environments as thermal vents in the ocean floor, sulphur springs and salt crystals. It is known that plasmids are found in most bacterial species. Plasmids are extrachromosomal pieces of circular DNA. Genes within the plasmid are often essential for the growth of the bacteria in certain extreme environments (FREIFELDER, David., Essentials of Molecular Biology. Jones and Bartlett Publishers, Inc. USA. 1985). Plasmids are known to be transferred frequently and rapidly amongst bacteria.

It is possible that any bacteria living in saline environments may contain plasmids allowing them to do so. These plasmids could then be transferred naturally into sulphide oxidising bacteria.

A general method for the adaptation of bacteria for use in the leaching of ores and concentrates has been described by the present applicant in co-pending International Patent Application PCT/AU02/00182, and the entire content thereof is incorporated herein by reference.

The preceding discussion of the background art is intended to facilitate an understanding of the present invention only. It should be appreciated that the discussion is not an acknowledgement or admission that any of the material referred to was part of the common general knowledge in Australia as at the priority date of the application.

Throughout the specification, unless the context requires otherwise, the word “comprise” or variations such as “comprises” or “comprising”, will be understood to imply the inclusion of a stated integer or group of integers but not the exclusion of any other integer or group of integers.

### DISCLOSURE OF THE INVENTION

In accordance with the present invention there is provided a method for the adaptation of bacteria for use in the leaching of ores and concentrates, the method characterised by the steps of:

a) Obtaining samples of bacteria exhibiting salt tolerance;

b) Combining bacterial samples from step a) with a stock bacterial culture known to have the ability to oxidise sulphide minerals; and

c) Grow the combined culture of step b) over time in saline conditions, whereby the resulting combined bacterial culture ultimately expresses both salt tolerance and the ability to oxidise sulphide minerals.

In accordance with the present invention there is further provided a method for the adaptation of bacteria for use in the leaching of ores and concentrates in generally saline conditions, the method characterised by the steps of:

a) Obtaining samples of water with salt tolerant bacteria;

b) Combining and growing bacterial samples of step a);

c) Combining a stock bacterial culture known to have the ability to oxidise sulphide minerals with a nutrient solution prepared from one or more of the samples of step a) and thereby beginning the adaptation of the stock bacterial culture to saline conditions; and

d) Combining a bacterial sample from step b) with a sample of culture from step c); and

e) Growing the combined samples of step d) and gradually increasing salinity, whereby the combined bacterial culture ultimately expresses both salt tolerance and the ability to oxidise sulphide minerals.

Preferably, the samples of water of step a) are used as a template to prepare ‘synthetic’ saline solutions which are in turn used to prepare the ‘synthetic’ saline nutrient solutions used in step c). Still preferably, the nutrient solution prepared from the sample having the lowest chloride ion concentration of the samples of step a) is used in Step c).

### BRIEF DESCRIPTION OF THE DRAWINGS

The present invention will now be described, by way of example only, with reference to one embodiment thereof and the accompanying drawing, in which:

FIG. 1 is a schematic diagram of a process for the adaptation of bacteria for use in the leaching of ores and concentrates in generally saline conditions in accordance with the present invention.

### BEST MODE(S) FOR CARRYING OUT THE INVENTION

Generally, the present invention is intended to collect bacteria capable of operating in saline waters and mix these bacteria with sulphide oxidising bacteria with the view that saline resistance would be transferred from the bacteria of the saline waters to the sulphide oxidising bacteria through the transfer of DNA from one species to another.

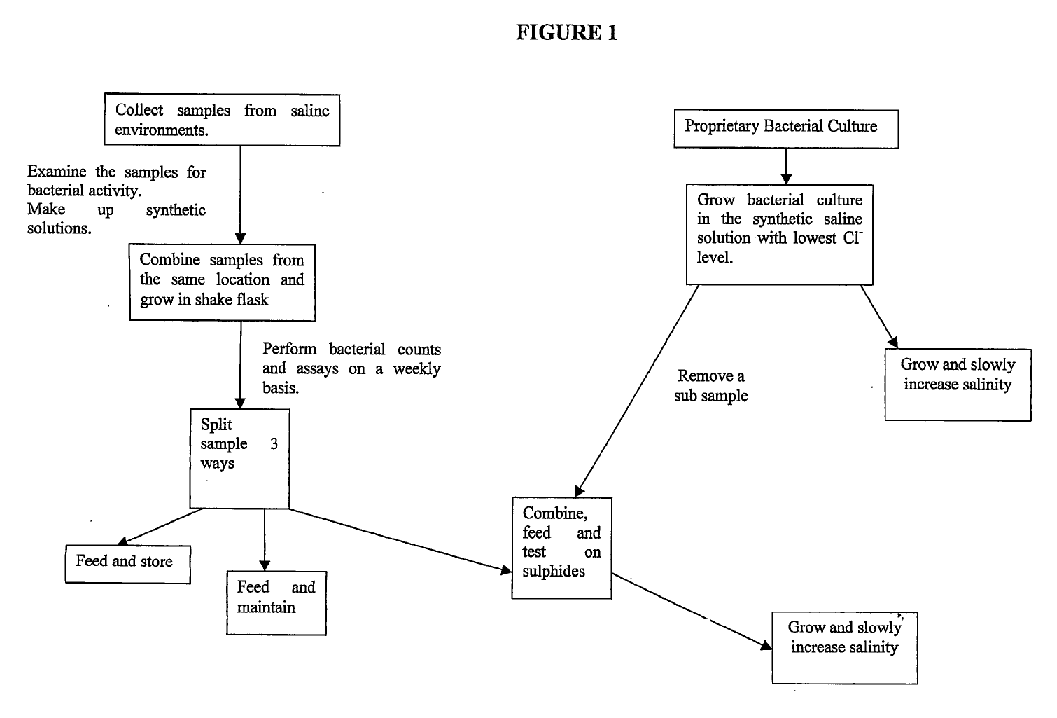

The method of the present invention will now be described with reference to an example and FIG. 1. The example is not intended to limit the generality of the foregoing description. FIG. 1 is to be read in conjunction with the following description.

The inventors determined that the best chance of the intended DNA transfer taking place would be between bacteria from similar environments. Naturally occurring bacteria samples were collected from “black smokers” from the sea. Black smokers are sulphide deposits found on the ocean floor. Examination of the smokers has revealed that they are teeming with bacteria. Additional samples were collected from puddles of process water within and around sulphide mines. Also from the mines, samples of ore and liquor from saline environments were collected, these samples consisted of liquors, sludges and dry solids. In all cases, samples of the water/liquor were removed from each location.

Each of the liquor/water samples collected were submitted for full ICP-OES (induced coupled plasma optical emission spectrometry) analysis, including sodium, chloride and TDS analysis, in order to determine the levels of the various salts within the samples. The pH's of the liquors and sludges were also determined.

The samples containing liquors were examined under a microscope and bacterial counts made.

The results from TDS and ICP analysis were used, much like a template, to make up ‘synthetic’ saline solutions for the appropriate samples using aquarium salts. Calculations were based on the levels of Cl⁻ rather than TDS. The synthetic saline solutions were used for making up ‘synthetic’ saline nutrient solutions, as it is often difficult to get sufficient water samples for testing.

Standard OK nutrient solutions are made using the synthetic saline solutions.

Any solid samples were split in half, one half was ground and used as a sulphide feed source for the indigenous bacterial samples from that location, the other half of the sample was not ground, as bacteria would have been destroyed from the shearing forces.

The unground solid sample was combined with the synthetic saline solution similar to its' indigenous salt water and the bacteria present on the solids grown up in shake flasks.

Solids and liquor samples from the same location were combined and placed in a shaker bath at 45° C. Yeast extract was added to these tests at a concentration of 0.1 g/L. Yeast extract provides nutrients for heterotrophic bacteria.

The pH of all the slurries was adjusted to the natural pH of the samples taken from the environments.

Every week the pH of the tests was adjusted down 0.5 of a unit until a pH of <2.0 is maintained. All pH adjustments were carried out through the addition of concentrated sulphuric acid.

The shake flasks were examined on a weekly basis for bacterial activity and the liquors sampled and assayed for metals reporting to solution.

A ‘stock’ bacterial culture capable of oxidising sulphide minerals was adjusted slowly to saline waters. The culture was grown in a sample of the synthetic nutrient solution with the lowest levels of Cl⁻ (approximately 13 g/L). The Cl⁻ levels were gradually increased over time, in some cases to chloride levels of 98 g/L over eight months.

Once the bacterial numbers from the indigenous salt samples were considered high enough (≧10⁷ cells/mL), the cultures were divided into three. A first sample fed and stored, a second sample scaled up, fed and maintained, and the third sample combined with an equal portion of the stock bacterial culture adapted to low levels of salinity as above.

The combined bacterial cultures were used as an inoculum for sulphide amenability testing. The volume of the test was made up to 3 L using the appropriate saline nutrient media, and the tests were conducted in standard stirred tank reactors at a temperature ranging between 40° C. to 55° C. The test was fed with a sulphide ore/concentrate and yeast extract added to a concentration of 0.1 g/L.

The test was monitored by assaying the levels of metals reporting to solution.

The transfer of genetic material from one bacterial species to another may take some time. However, the resulting bacterial culture is capable of both growing in saline environments and oxidising sulphide minerals.

The salinity of the test may be increased with each successive scale up to chloride levels of at least 40 to 55 g/L, and up to about 98 g/L, or to TDS levels of at least 80,000 to 90,000 ppm, and up to about 200,000 ppm. The inventors envisage that the chloride and TDS levels may be able to be taken higher than these levels if required.

Modifications and variations such as would be apparent to the skilled addressee are considered to fall within the scope of the present invention.

1. A method for the adaptation of bacteria for use in the leaching of ores and concentrates, the method characterised by the steps of:
a) Obtaining samples of bacteria exhibiting salt tolerance;
b) Combining bacterial samples from step a) with a stock bacterial culture known to have the ability to oxidise sulphide minerals; and
c) Grow the combined culture of step b) over time in saline conditions, whereby the resulting combined bacterial culture ultimately expresses both salt tolerance and the ability to oxidise sulphide minerals.

2. A method according to claim 1, wherein the bacterial samples of step a) are obtained with water samples, these water samples being used as a template to prepare ‘synthetic’ solutions which are then in turn used to prepare nutrient solutions for the stock bacterial culture of step b).

3. A method according to claim 1, wherein the bacterial samples of step a) are obtained from at least two locations and subsequently combined and grown, this combined culture then being combined with the stock bacterial culture in step b).

4. A method for the adaptation of bacteria for use in the leaching of ores and concentrates in generally saline conditions, the method characterised by the steps of:
a) Obtaining samples of water with salt tolerant bacteria;
b) Combining and growing bacterial samples of step a);
c) Combining a stock bacterial culture known to have the ability to oxidise sulphide minerals with a nutrient solution prepared from one or more of the samples of step a) and thereby beginning the adaptation of the stock bacterial culture to saline conditions;
d) Combining a bacterial sample from step b) with a sample of culture from step c); and
e) Growing the combined samples of step d) and gradually increasing salinity, whereby the combined bacterial culture ultimately expresses both salt tolerance and the ability to oxidise sulphide minerals.

5. A method according to claim 4, wherein, the samples of water of step a) are used as a template to prepare ‘synthetic’ saline solutions which are in turn used to prepare the ‘synthetic’ saline nutrient solutions used in step c).

6. A method according to claim 5, wherein the nutrient solution prepared from the sample having the lowest chloride ion concentration of the samples of step a) is used in Step c).

7. A method according to claim 6, wherein the lowest chloride concentration is about 13 g/L.

8. A method according to claim 4, wherein salinity levels are increased to levels of at least 40 g/L in step e).

9. A method according to claim 4, wherein salinity levels are increased to levels of at least about 98 g/L in step e).

10. A method according to claim 4, wherein the salinity levels are increased over a period of about eight months in step e).

11. A method according to claim 4, wherein the levels of total dissolved solids (TDS) is increased to at least about 80,000 ppm.

12. A method according to claim 4, wherein the levels of total dissolved solids (TDS) is increased to at least about 200,000 ppm.

13. (CANCELED)

14. (CANCELED)

15. (CANCELED)
